(19) United States
(12) Patent Application Publication
(10) Pub. No.: US 2002/0169432 A1
(43) Pub. Date: Nov. 14, 2002

(54) Absorbent garment with expandable absorbent element

(76) Inventors: David A. Fell, Neenah, WI (US); Paul T. Van Gompel, Hortonville, WI (US)

Correspondence Address: BRINKS HOFER GILSON & LIONE, P.O. Box 10395, Chicago, IL, 60610, US

(21) Appl. No.: 09/855,182

(22) Filed: May 14, 2001

(51) Int. Cl. A61F 13/15

(52) U.S. Cl. 604/385.14

(57) ABSTRACT

An absorbent garment comprises a chassis and an absorbent element. The absorbent element is fixedly connected to the chassis at a first location and is detachably connected to at least one of the absorbent element and the chassis at a second location. The absorbent element comprises an absorbent material that expands from at least a first condition to a second condition. The absorbent element detaches from at least one the absorbent element or the chassis at at least a portion of the second location when the absorbent material expands to the second condition. A method for using the absorbent garment also is provided, together with a method for assembling the absorbent garment.

### BACKGROUND

The present invention relates generally to absorbent garments, and in particular, to an absorbent garment having an expandable absorbent element and to methods for making and using such a garment.

Absorbent garments, and in particular disposable absorbent garments, typically include a bodyside liner and an absorbent element, which typically includes an absorbent material. Often, such garments include a body chassis for supporting the absorbent element, including for example incontinence undergarments, which are typically configured with a self-supporting waist band, or diapers and the like, which can be secured on the user with tabs, belts and the like. Typically, the absorbent element is disposed between the body chassis and the user, such that any swelling or expansion of the absorbent material, caused by the excretion or exudation of liquids and other insult from the user, acts against the body chassis. As a result, the expansion of the absorbent material can cause the body chassis to move away from the body of the user, which can lead to leakage of the liquids and insult. Alternatively, or in addition, the expansion of the absorbent material can force the absorbent element against the body of the user so as to cause some level of discomfort.

### SUMMARY

Briefly stated, in one aspect, the invention is directed to an absorbent garment comprising a chassis and an absorbent element. The absorbent element is fixedly connected to the chassis at a first location and is detachably connected to the chassis at a second location. The absorbent element comprises an absorbent material that is expandable from at least a first condition to a second condition. The absorbent element detaches from the chassis at at least a portion of the second location when the absorbent material expands to the second condition.

In a preferred embodiment, the absorbent element further comprises an outer cover supporting the absorbent material. Also in a preferred embodiment, the first location preferably includes at least one primary bond region and the second location preferably includes at least one secondary bond region, with each of the bond regions extending longitudinally along at least a portion of the outer cover. Alternatively, or in addition, the bond regions can extend laterally along portions of the absorbent element.

In a more preferred embodiment, the primary bond region comprises a pair of laterally spaced, parallel and longitudinally extending primary bond regions, and the secondary bond regions comprise a pair of laterally spaced, parallel and longitudinally extending secondary bond regions. In one preferred embodiment, the secondary bond regions are positioned outboard and on opposite sides of the primary bond regions in a parallel relationship therewith.

In another aspect of the invention, the absorbent element is not detachably connected to the body chassis with a secondary bonding, but rather is maintained proximate the body chassis in an overlapping loose configuration without any secondary bonding. In this embodiment, the absorbent element simply moves away from the body and the body chassis as it absorbs various bodily exudates.

In yet another aspect, the absorbent element can be detachably connected to itself at various second locations. For example, the cover sheet of the absorbent element can be folded back on itself with a secondary bond located between the folds of the cover. In one preferred embodiment, the primary bond regions are located along the outer peripheral side edges of the cover sheet, while the secondary bond regions are located inboard of the primary bond regions between the folds of the cover sheet.

In one preferred embodiment, the chassis comprises an outer cover, which is preferably extensible, and a top sheet. Preferably, the absorbent element is disposed between the outer cover and the top sheet, and is attached to the top sheet. In one embodiment, both the cover sheet and the absorbent material of the absorbent element are attached to the top sheet.

In an alternative preferred embodiment, the chassis comprises a pair of body panels, including a front and back body panel. The absorbent element extends between and connects the body panels. The absorbent element can be connected to either the bodyside or the garment side of the body panels.

In another aspect of the invention, the absorbent material is preferably folded. Preferably, the absorbent material includes an opening between the folds that is exposed to the body of the user and the liquids being introduced by the user.

In yet another aspect of the invention, the absorbent material comprises a plurality of disconnected layers stacked one on top of the other, with each layer preferably comprised of one or more strips. Preferably, one or more strips in an uppermost layers form an opening therebetween that is exposed to the body of the user and the liquids being introduced by the user.

In yet another aspect of the invention, the absorbent material comprises a plurality of barrier members attached to the outer cover and extending laterally inboard therefrom and a base layer disposed beneath the barrier members. In operation, the barrier members swing downwardly as the absorbent element absorbs liquids and as the base layer expands outwardly away from the body of the user.

In another aspect of the invention, a method of absorbing a bodily exudate excreted from a user with an absorbent garment includes securing the chassis to a body of a user, insulting the absorbent material with the bodily exudate and thereby causing said absorbent material to expand. The absorbent element is disconnected from the chassis, or from itself, at the second location as the absorbent material expands while the connection between the absorbent element and the chassis is maintained at the first location. In an alternative embodiment, where there is no secondary boding, the absorbent element is initially held close to the body chassis in the dry state, but moves away from the chassis as it absorbs the exudates and swells.

In yet another aspect of the invention, a method of assembling an absorbent garment includes bonding the absorbent element with a primary bond to the chassis along at least one primary bond region, and bonding the absorbent element to the chassis or to itself with a secondary bond along at least one secondary bond region, wherein the secondary bond is weaker than the primary bond.

The present invention provides significant advantages over other absorbent garments and methods for the use and manufacture thereof. For example, the absorbent element is attached to the body chassis such that it can act independently therefrom. In particular, as the absorbent material expands or swells in response to the introduction of various liquids and the like, the absorbent element, which is preferably attached to the outside of the body chassis, can move away from the body chassis rather than forcing the body chassis away from the user or from forcing the absorbent element into the user. As the absorbent material expands, the secondary bonds, which are preferably weaker than the primary bonds, are broken so as to allow the absorbent element to swell. At the same time, the primary bonds ensure that the absorbent element maintains its attachment to the chassis while at the same time preventing the liquids from escaping from between the absorbent element and the chassis. Hence, a good fit and gasketing of the garment are maintained and which thereby improve the leakage protection of the garment and the resulting dignity and comfort of the user.

In addition, the configuration of the garment, in one preferred embodiment, does not require the bodyside liner to be attached to either the chassis or the absorbent element, thereby simplifying the manufacturing process and reducing the costs thereof.

The disconnected layers of absorbent material also provide significant advantages. In particular, each layer, and/or each strip of each layer, can be constructed of different materials having different absorptive capacities. In addition, the process of folding the absorbent material is also eliminated.

The folded absorbent material also provides significant advantages. In particular, the folds increase the effective surface area available for liquid intake and absorption. As the liquid enters the folded structure, it can flow along the layers of material and be absorbed more quickly then if a single thickness or layer of material is used. In addition, the use of a folded absorbent material allows for the use of thin composite materials for the absorbent that can be unwound from rolls with longer running times between roll changes compared to thicker materials. Moreover, the presence of folds in the absorbent material improves the wicking of liquid.

The present invention, together with further objects and advantages, will be best understood by reference to the following detailed description taken in conjunction with the accompanying drawings.

### BRIEF DESCRIPTION OF THE DRAWINGS

Many of the features and dimensions portrayed in the drawings, and in particular the presentation of sheet material web thicknesses and the like, have been somewhat exaggerated for the sake of illustration and clarity.

FIG. 1 is a plan view of an absorbent element taken from the bodyside thereof.

FIG. 2 is a partial cross-sectional view of one preferred embodiment of an absorbent garment in an unexpanded condition.

FIG. 3 is a partial cross-sectional view of an alternative preferred embodiment of an absorbent garment in an unexpanded condition.

FIG. 4 is a partial cross-sectional view of the absorbent garment shown in FIG. 3 in an expanded condition.

FIG. 5 is a partial cross-sectional view of another alternative preferred embodiment of an absorbent garment.

FIG. 6 is a partial cross-sectional view of another alternative preferred embodiment of an absorbent garment.

FIG. 7 is a perspective view of one embodiment of an absorbent material.

FIG. 8 is a perspective view of an absorbent garment.

FIG. 9 is a partial cross-sectional view of another alternative preferred embodiment of an absorbent garment.

FIG. 10 is a partial cross-sectional view of another alternative preferred embodiment of an absorbent garment.

FIG. 11 is a partial cross-sectional view of an alternative preferred embodiment of an absorbent garment in an unexpanded condition.

FIG. 12 is a partially cut away, bodyside plan view of an absorbent garment.

FIG. 13 is a cross-sectional view of an absorbent garment taken along line 13-13 of FIG. 12.

FIG. 14 is an alternative cross-sectional view of an absorbent garment taken along line 14-14 of FIG. 12.

FIG. 15 is a bodyside plan view of an absorbent garment.

FIG. 16 is a cross-sectional view of an absorbent garment taken along line 16-16 of FIG. 15.

FIG. 17 is an alternative cross-sectional view of an absorbent garment taken along line 17-17 of FIG. 15.

FIG. 18 is a bodyside plan view of an absorbent garment.

FIG. 19 is a cross-sectional view of an absorbent garment taken along line 19-19 of FIG. 18.

FIG. 20 is an alternative cross-sectional view of an absorbent garment taken along line 20-20 of FIG. 18.

FIG. 21 is a schematic illustration of a uniform detachable bond.

FIG. 22 is a stress/strain graph of the bond shown in FIG. 21.

FIG. 23 is a schematic illustration of a gradient detachable bond.

FIG. 24 is a stress/strain graph of the bond shown in FIG. 23.

### DETAILED DESCRIPTION OF THE PRESENTLY PREFERRED EMBODIMENTS

It should be understood that the term “longitudinal,” as used herein, means of or relating to length or the lengthwise direction, and in particular, the direction running between the front and back of the user. The term “laterally,” as used herein means situated on, directed toward or running from side to side, and in particular, a direction running from the left to the right of a user. The terms “upper,” “lower,” “inner,” and “outer” as used herein are intended to indicate the direction relative to the user wearing an absorbent garment 2 over the crotch region, while the terms “inboard” and “outboard” refer to the directions relative to a centerline 30 of the garment. For example, the terms “inner” and “upper” refer to a “bodyside,” which means the side closest to the body of the user, while the terms “outer” and “lower” refer to a “garment side.”

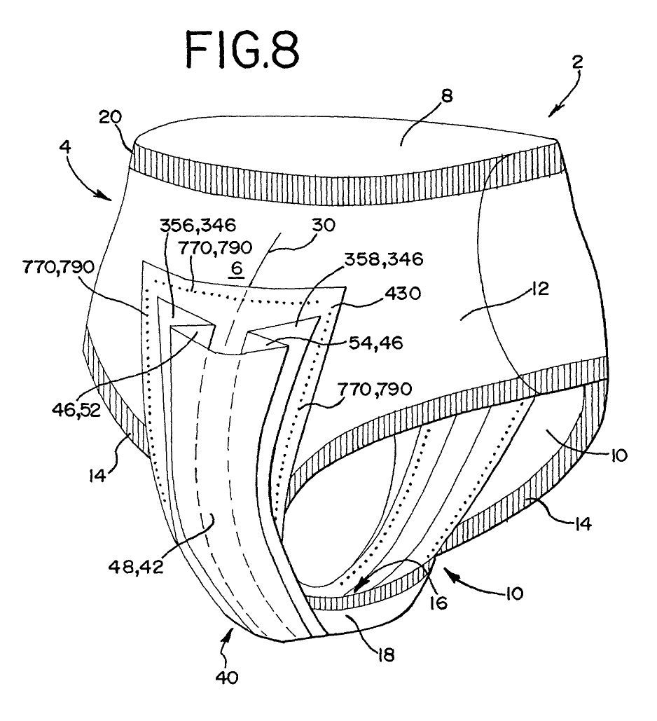

Referring to FIG. 8, one embodiment of an absorbent garment includes a bodyfit chassis. The chassis 4 includes form-fitting body coverage elements, including front and rear body panels 6, 8 and a crotch region 18 extending between the body panels. The body chassis maintains a comfortable snug fit against the body of the user. As best shown in FIGS. 2-6, the chassis 4 has a longitudinally extending opening 16 formed along the crotch region of the chassis. The chassis has an inner, bodyside surface 10 and an outer surface 12. Preferably, the chassis includes elastic regions 14 formed along the edges of the crotch region and around the leg openings, so as to form a gasket with the user's crotch and legs. It should be understood, for the purposes of this invention, that the chassis can take many forms, including for example, the preferred pant-like or underwear type undergarment shown in FIG. 8, which includes a self supporting waistband 20 extending circumferentially around the waist of the user, or alternatively a diaper or like garment, which is secured around the user with various fastening means or devices known by those of skill in the are, including for example and without limitation tabs, belts and the like.

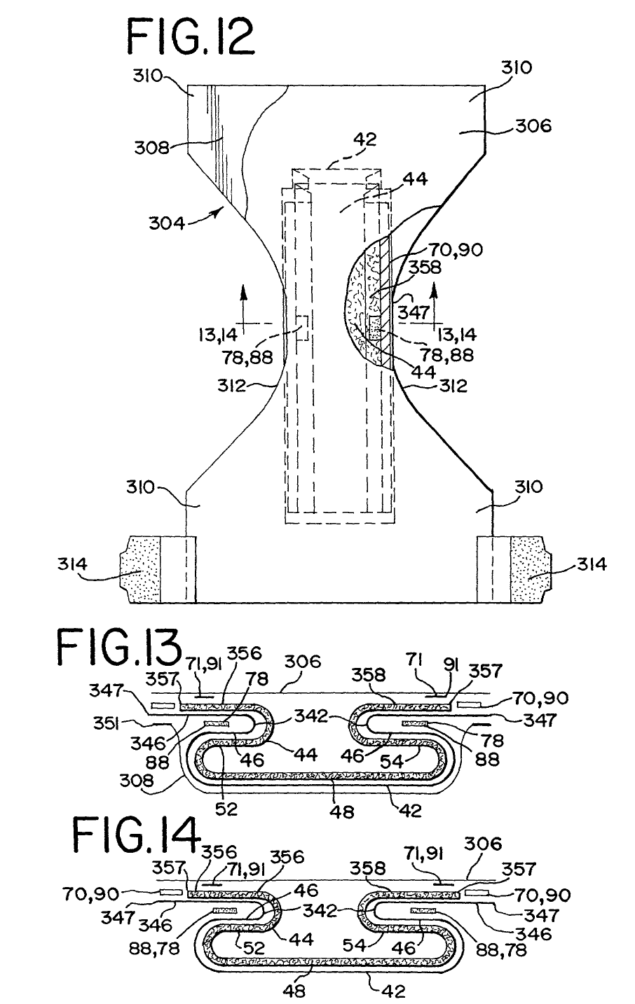

For example, in one alternative embodiment shown in FIGS. 12-13, a body chassis 304 includes a liquid permeable top sheet 306 secured to an extensible outer cover 308. The outer cover can also be referred to as backsheet, which can be liquid permeable or impermeable, depending on whether an additional backsheet, or barrier sheet, is provided. Alternatively, as shown in FIG. 14, the body chassis includes only one or more top sheets, without the extensible outer cover. Each of the top sheet and outer cover has ear portions 310 extending laterally outward at each end of the absorbent garment, and concave shaped peripheral side edges 312 extending between the ear portions. Preferably, elastic members are secured along the peripheral side edges to form a gasket along the leg or crotch region of the user. Fastening tabs 314 are attached to the ear portions on one end of the garment. The fastening tabs can comprise hooks and/or loops, such as a VELCRO® fastening system, or can have adhesive or other bonding agents applied to one surface thereof. Alternatively, the fastening tabs can include buttons, snaps, ties or other know fastening means or devices. When the garment is secured to the body of the user, the fastening tabs 314 secured to the ear portions on one end of the garment engage or are otherwise connected to the ear portions on the opposite end of the garment. When secured in this way, openings are formed on each side of the garment along the concave peripheral edge and are shaped to receive the legs of the user.

In another alternative embodiment shown in FIGS. 15 and 18, a body chassis 404 includes a front and back panel 406, 408, with one of the panels, preferably the back panel, having a pair of tabs 314 secured thereto. Each panel includes ear portions 310 and opposite ends 410, 416 and 412, 414, with the ends 410, 412 spaced apart to form an opening 420 therebetween. The absorbent element 40 spans the opening 420 and extends between and connects the front and back body panels. In the embodiment of FIG. 15, the absorbent element is connected to the inner body side of the front and back panels, while in the embodiment of FIG. 18, the absorbent element is connected to the outer, garment side of the front and back panels. The front and back body panels can include various layers or sheets, including an extensible outer cover.

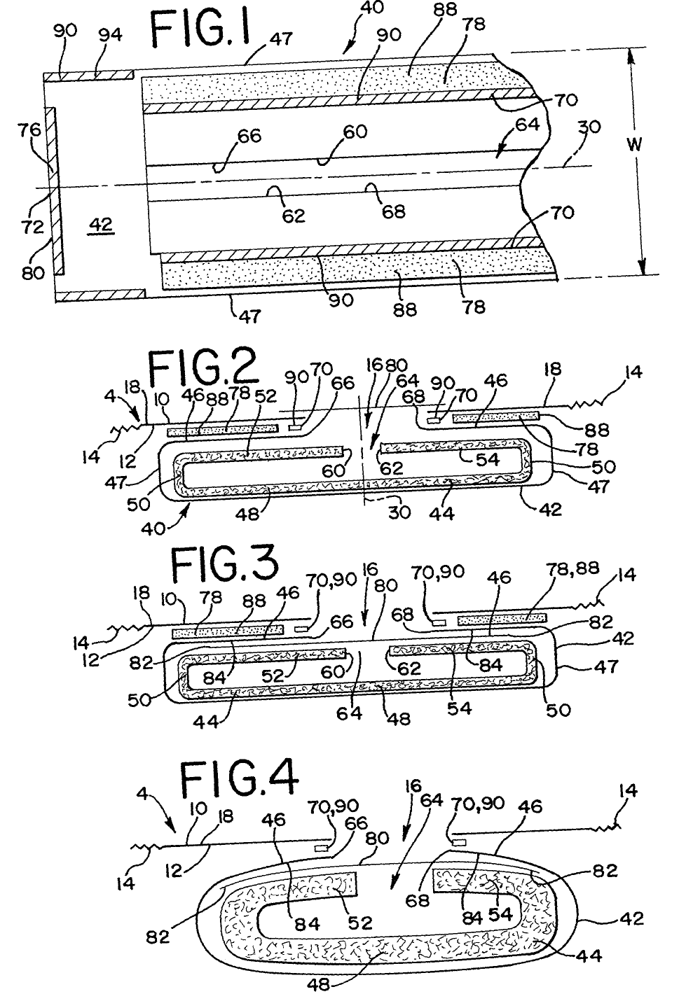

As best shown in FIGS. 1 and 2, a first embodiment of an absorbent element 40 is shown as including a backsheet 42, which is preferably liquid impermeable and which is otherwise referred to as a cover sheet or barrier sheet, and an absorbent material 44. The backsheet 42 preferably includes opposite folds 46 that are folded laterally inwardly over a portion of the absorbent material 44. The absorbent material can be simply supported by the backsheet without any attachment therebetween, or it can be bonded or otherwise secured to the backsheet. The absorbent element has opposite sides or side edges 47.

The absorbent material 44 can be any material that tends to swell or expand as it absorbs exudates, including various liquids and/or fluids excreted or exuded by the user. For example, the absorbent material can be made of airformed, airlaid and/or wetlaid composites of fibers and high absorbency materials, referred to as superabsorbents. Superabsorbents typically are made of polyacrylic acids, such as FAVOR 880 available from Stockhausen, Inc. of Greensboro, N.C. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. In addition, various foams, absorbent films, and superabsorbent fabrics can be used as an absorbent material. Various acceptable absorbent materials are disclosed in U.S. Pat. No. 5,147,343 for Absorbent Products Containing Hydrogels With Ability To Swell Against Pressure, U.S. Pat. No. 5,601,542 for Absorbent Composite, and U.S. Pat. No. 5,651,862 for Wet Formed Absorbent Composite, all of which are hereby incorporated herein by reference. Furthermore, the properties of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom.

In one preferred embodiment, a folded absorbent material is made of fibrous absorbent materials with relatively high internal integrity, including for example one made with thermoplastic binder fibers in airlaid absorbents, e.g., pulp, bicomponent binding fibers, and superabsorbents, which have higher densities in the folded regions. The higher density and resulting smaller capillary size in these regions promotes better wicking of the liquid. Better wicking, in turn, promotes higher utilization of the absorbent material and tends to result in more uniform swelling throughout the absorbent material as it absorbs the liquid.

Referring to FIGS. 1-4, 6 and 7, one preferred embodiment of the absorbent material comprises a first fold 48 or layer having opposite folding edges 50, and a second and third fold 52, 54 extending laterally inward from the folding edges 50 in an overlying relationship with the first fold 48. The folds form a folding edge or pleat at the outboard edges of the absorbent material. The absorbent material preferably has a greater longitudinal extent, or length (L), than its lateral extent or width (W), which is defined in this embodiment by the width of the first fold 48. Of course, it should be understood that the absorbent material can made of a single layer or a monolithic unfolded material, or a plurality of disconnected layers.

For example, as best shown in FIG. 9, the absorbent material 144 comprises a two layer composite, with a first base layer 148 and a second layer stacked thereon. The second layer 150 preferably includes a pair of spaced apart strips 152, 154, which preferably form an opening 156 therebetween. The second layer 150 is preferably not attached to the first layer 148, although it should be understood that the two layers could be attached to each other by bonding and the like. The strips 152, 154 making up the second layer can be made of different materials than the underlying base layer 148, and from each other, so as to provide different absorptive capacities for each of the strips and/or layers. It should be understood that the stack of layers can be made up of a plurality of layers, including more than two layers, and that each layer, including the base layer, can be made of one or more strips, e.g., three strips. As such, the embodiment of FIG. 9, with its two layers, and its two strips making up the second layer, is meant to be illustrative rather than limiting. It should be understood that the term “plurality” means two or more. By providing disconnected layers, the absorbent material composite can be made without having to fold a sheet material web.

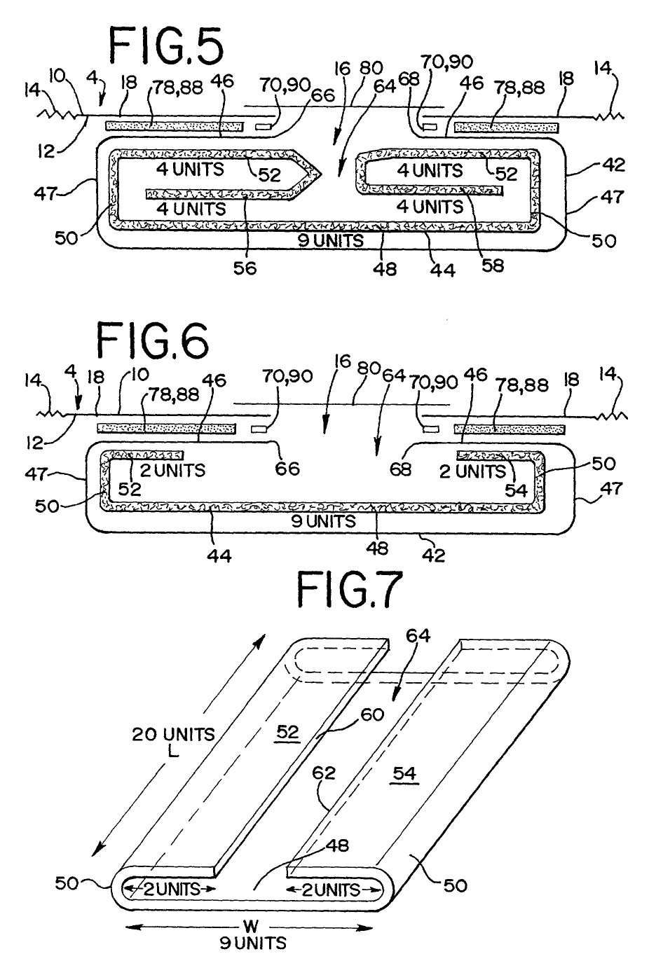

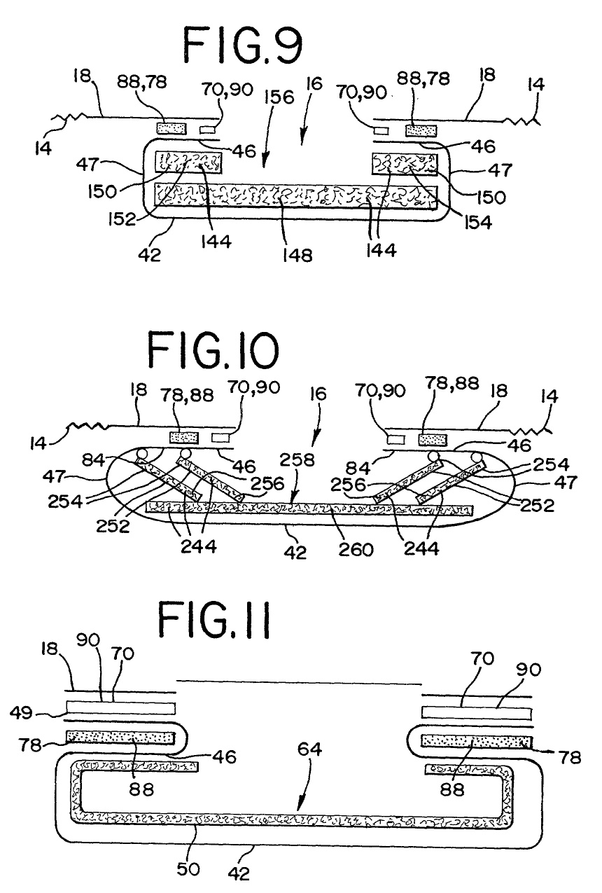

Referring to FIG. 10, the absorbent material 244 includes a plurality of barrier members 252, or strips, having an outboard lateral edge 254 hingedly connected to an inner surface 84 of the cover sheet 42, which acts as a support member for the barrier members. Alternatively, the barrier layers can be directly connected to the outer surface of the body chassis, preferably with the cover sheet also attached to the body chassis at first and second bond locations. The barrier strips 252 extend laterally inward and terminate at a free edge 256. The free edges of the innermost barrier strips form an opening 258 therebetween. Preferably, a base layer 260 is disposed beneath the barrier strips 252, such that the layers 260, 252 form a stack of layers. In operation, the liquid is introduced to the base layer through the opening 258. The base layer 260 expands as it absorbs liquid, as do the barrier strips, which swing away from the body of the user about the hinged connection. It should be understood that the number of barrier members can be increased or decreased as desired, e.g., to increase the surface area of the absorbent material. In addition, it should be understood that additional layers or strips can be stacked on the base layer, as shown for example in FIG. 9, and can be used in combination with the barrier members. Alternatively, the base layer can include various folds, as shown for example in FIGS. 2 and 5, with the base layer again being used in combination with the barrier members. It also should be understood that the free edge of the barrier layers can extend laterally outward from the hingedly connected edge thereof.

The folded and/or stacked absorbent material has additional surface area of absorbent material available for liquid intake as compared with a single layer structure. The amount of surface area is correlated with a fold factor (Ff), which is equal to the sum of the widths of the various folds, or layers and strips, divided by the width of the widest layer, which typically corresponds to the overall width of the absorbent material, for example, to the width of the first fold in FIGS. 1-4, 6, and 7, or to the sum of the widths of the various layers, for example, the three strips in FIG. 9 or the five strips of FIG. 10.

In one preferred embodiment, shown in FIGS. 1-4, the width of the second and third folds is about 4 units, while the underlying first fold has a width of about 9 units. It should be understood that the term “units” can be any unit of measurement, and that it is not the precise length of the unit that is important, but rather the relationship of the length of the various folds relative to the other folds. Indeed, the Ff is unitless and does not depend on any particular unit of measurement. The embodiment shown in FIGS. 1-4 has a fold factor Ff=(4+4+9)/9=1.9. A single fold or layer absorbent material would have a fold factor of 1.0. It is preferred that the fold factor be greater than 1.0.

In the alternative preferred embodiment of FIGS. 6 and 7, the second and third folds have a width of about 2 units, thereby yielding a fold factor Ff=(2+2+9)/9=1.4.

In yet another alternative embodiment, shown in FIG. 5, the absorbent material further comprises a fourth and fifth fold 56, 58 extending laterally outward or outboard from an inner edge 60, 62 of the second and third folds 52, 54 respectively, in an underlying relationship therewith so as to form a pair of pleats or folding edges. Each of the second, third, fourth and fifth folds 52, 54, 56, 58 preferably have a width of about four units. As such, the fourth and fifth folds are sandwiched between the first fold 48 and the second and third folds 52, 54 respectively. In this embodiment, the absorbent material has a fold factor Ff=(4+4+4+4+9)/9=2.8.

In yet another alternative embodiment, the absorbent material shown in FIG. 5 can be made discontinuous from left to right, with two longitudinally extending absorbent elements having various folds and with the elements separated, preferably along the centerline of the absorbent composite.

In yet another alternative embodiment, shown in FIGS. 13, 14, 16, 17, 19 and 20, the absorbent material, or retention portion, comprises a fourth and fifth fold 356, 358 extending laterally outward or outboard from an inner edge 60, 62 of the second and third folds 52, 54 respectively, in an overlying relationship therewith. In this embodiment, a barrier layer, or cover sheet, is interfolded with the absorbent material. In particular, the barrier layer comprises opposite folds 346 extending laterally outward or outboard from an inner edge of the folds 46 respectively, in an overlying relationship therewith so as to form a folding edge or pleat 342. The opposing faces of the folds 346, 46 are in preferably in contact as they lie between or are nested in the folds 356, 52 and 358, 54 of the absorbent material. Preferably, an outboard edge 347 of the barrier layer folds 346 extends laterally outboard beyond an outboard edge 357 of the absorbent material on each side of the garment, such that the barrier layer can be attached to one or more of the topsheet or body panels.

As the fold factor Ff increases, the surface area of the absorbent material available for liquid intake also increases. Indeed, the fold factor can also be calculated using various areas of the absorbent material. For example, as shown in FIG. 7, an absorption area Aa is equal to the sum of the various widths of the folds times the length (L) of the absorbent material, while the facing area Fa is equal to the length (L) of the absorbent material times the overall width (W) thereof. Thus, in the embodiment shown in FIGS. 6 and 7, Aa=(9+2+2)×20=260 units² and Fa=20×9=180 units². The fold factor Ff=Aa/Fa=1.4.

The facing area Fa thus refers to the area calculated from the gross dimensions of the folded absorbent material. The actual total area of the absorbent material surface area available for liquid intake is obtained by multiplying the facing area Fa by the fold factor Ff. For example, in the embodiment shown in FIG. 3, which has a fold factor Ff of 1.4, the available absorption area is 40% greater than the facing area Fa. Likewise, the embodiments of FIGS. 2 and 5 have available areas of 90% and 180% greater than the facing areas Fa respectively. Indeed, these percentage increases are further underestimated since both sides of certain of the folds are available for liquid intake, especially as the structure swells or expands.

Athough the opening defined between between the folds, or between the strips defining a layer of absorbent material, has been shown as facing toward the body of the user, it should be understood that absorbent material, or layers thereof, could be oriented such that the opening opens outwardly away from the user, for example, when rapid intake of liquid is not required.

In each of the embodiments of FIGS. 1-6 and 8, the absorbent element 40 is preferably attached to the outer, garment side surface 12 of the chassis 4, with the upper folds 52, 54 facing the opening 16 formed in the chassis. Preferably, the inboard edges 60, 62 of the folds 52, 54 are spaced apart to provide an opening 64 therebetween so as to allow the liquid to be received by the lower fold 48 and migrate into the areas between the folds. In the embodiments of FIGS. 1-6, each of the peripheral inboard edges 66, 68 of the cover sheet is preferably fixedly attached to the chassis 4 along a longitudinally extending location 70 adjacent an edge of the chassis that forms the opening 16 therein. In the embodiment of FIG. 8, the topsheet is fixedly attached to the body chassis with primary bonds 790 along a location 770, such that the absorbent composite is disposed over the opening 16, and the retention portion is secured to the topsheet with both primary and secondary bonds 90, 88. It should be understood that the term “location” means any point, line, or region, which region defines an area, or any combination thereof, including a plurality of such points, lines and regions. It should further be understood that the term “fixedly” means an attachment that is not intended to be removed or disengaged during the normal use and operation of the absorbent garment, and in particular, in response to the expansion of the absorbent element away from the user's body.

Preferably, in the embodiments of FIGS. 1-6, the cover sheet 42 is attached to the chassis along the first location 70 by bonding a top side of the folds 46 of the cover sheet 42 to the outer surface 12 of the chassis with a primary bond 90. The primary bonds can be made using hot melt adhesives, such as Findley HX2717-22, sonic bonds, thermal bonds, and/or mechanical bonds, such as by sewing or engaging hook and loop-type fastening systems. In the latter embodiment, the primary bonds are made fixedly detachable, meaning that the absorbent element does not detach in response to the applied expansion forces of the absorbent element, but can be completely removed by the user from the body chassis after use and thereafter replaced with another element. Alternatively, the absorbent material can be removed from the cover sheet, which is fixedly, detachably connected to the body chassis, and a replacement absorbent material insert, comprised of one or more layers or folds, can be removeably disposed in the cover sheet, which can thereafter be fixedly, reattached to the body chassis. The primary bond, whether detachable or not, is required to be greater than any forces applied by an expanding absorbent material against the body of the user, which tends to push the cover sheet away from the body of the user.

Further primary bond regions 72, 74 are preferably formed between end portions 76 of the cover sheet 42 and the chassis, on opposite ends of the opening 16 formed the chassis. The additional primary bond locations 72, 74, which are formed along the end edge and lateral of the edges backsheet respectively, maintain a firm attachment of the absorbent element 40 to the chassis 4 while at the same time allowing a hinging effect during the expansion of the absorbent material, which is explained below. It should be understood that other primary bonds can be applied in a laterally extending direction, or in other various patterns as desired, including for example various curvilinear, checked and/or grid patterns.

The cover sheet 42 is further detachably connected to the chassis along a pair of second locations 78 spaced laterally outboard from the first locations 70. The second locations 78 preferably are formed as a region having a greater width than the first location 70, and which preferably extend outboard from the first location to the outboard edge of the absorbent element. The pair of second locations 78 preferably extend longitudinally along a portion of the outer cover 42 and chassis 4 in a parallel relationship with the pair of first locations 70. It should be understood that other secondary locations can extend laterally, or can comprise various other patterns as desired, including for example various curvilinear, checked and/or grid patterns.

The cover sheet 42 is preferably attached to the chassis at the second locations 78 with a secondary bond 88, the strength of which is preferably weaker than the strength of the primary bond attaching the cover sheet to the chassis at the first locations, and also is preferably weaker than the expansion forces applied to the cover sheet by the absorbent material as it expands away from the body of the user. In essence, the strength of the detachable secondary bonds is less than the force applied by the weight of the insulted absorbent composite and any force exerted by the swelling material. Conversely, the strength of the detachable secondary bonds is greater than the weight of the dry absorbent composite.

In this way, the bond strength of the detachable secondary bonds means the total force required to detach the secondary bond. The function of the secondary bonds is to provide increased surface area and volume as the absorbent material intakes fluid and other bodily exudates and expands.

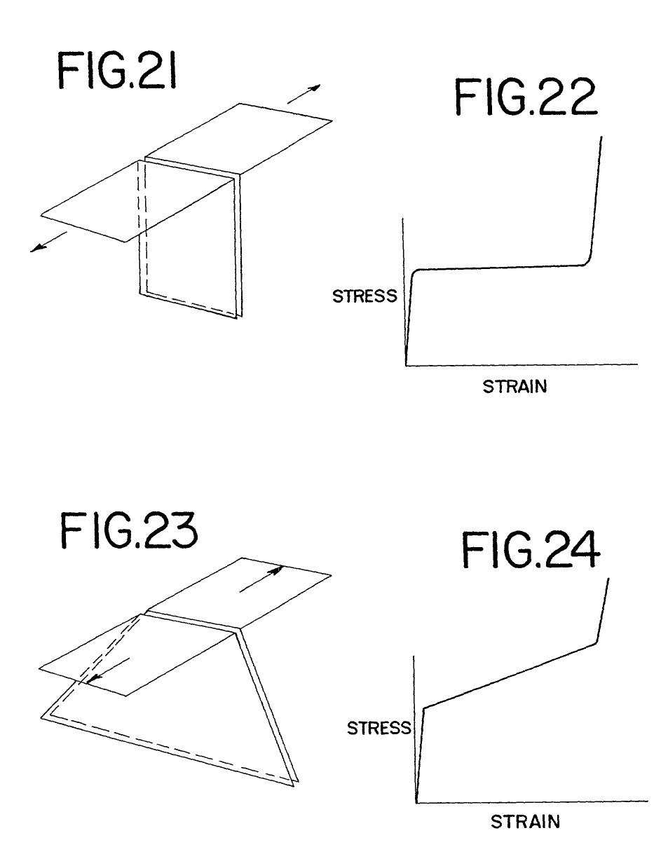

Referring to FIGS. 21-24, the secondary bond can be configured as a uniform detachable bond or as a gradient detachable bond. With the uniform detachable bond, the bond will become detached at a relatively constant stress until complete failure thereof as represented in FIG. 22. The uniform detachable bond can be achieved by providing a relative constant amount of bonding area as one moves away from the initial site of detachment, as shown for example in FIG. 21.

Alternatively, a gradient bond requires an increasing stress, or corresponding force, to further detach portions of the bond. In essence, as the total force applied by the weight of the insulted absorbent composite plus any forces exerted by the swelling material reaches an initial detachable bond strength, the secondary bond will begin detaching. Further detachment occurs as additional liquid or exudates is added so as to thereby increase the weight of the absorbent material and the swelling forces. Eventually, the weight of the absorbent material and the swelling forces will effect a complete detachment of the secondary bond as represented in FIG. 24. The gradient detachable bond can be achieved by increasing the amount of bonding area, e.g., by increasing the length of the bond at the point of detachment, as one moves away from the initial site of detachment, as shown for example in FIG. 23.

Alternatively, as shown in FIG. 11, the cover sheet may include an additional, uppermost fold 49 that is folded back over fold 46, or a first portion of the absorbent element. The uppermost fold 49, or second portion of the absorbent element, includes an upper surface that is secured to the outer surface of the chassis along a first location 70 with a primary bond 90. A secondary bond 88 can then be applied at a second location 78 between the uppermost fold 49 and the fold 46, such that the cover sheet, and the absorbent element, is detachably connected to itself. It should be understood that any of the various absorbent material configurations and liner configurations can be used in this embodiment. In addition, it should be understood that the cover sheet can be detachably connected to both itself and the body chassis at a second location, for example, if the uppermost fold is shortened such that that fold 46 overlaps a portion of the uppermost fold and a portion of the chassis.

Alternatively, as shown in FIGS. 12-14, a secondary bond 88 is applied at a second location 78 between the opposing faces of the folds 346, 46 of the cover sheet, or barrier layer, such that the absorbent element is detachably connected to itself. Preferably, the second location 78 and the secondary bond 88 comprise a small rectangular patch located in the crotch region of the garment. It is in this location that the force of the insult or exudates is the greatest and the location where the barrier sheet has the highest amount of folding or compression at the peripheral side edges from the legs of the user. Of course, it should be understood that second location can be positioned on either side of the crotch region. Alternatively, the second location and secondary bond can be formed as a continuous line or region of attachment or a series of dots or dashes extending longitudinally along the length of the garment between the folds of the barrier sheet.

Preferably, the outboard edge 347 of the folds 346 of the cover sheet or barrier sheet that extends beyond the outboard edge 357 of the absorbent material on each side of the garment is secured to the top sheet 306 or other member of the body chassis with a primary bond 90 at a first location 70, which is preferably located outboard the second location. The folds 356, 358 of the retention portion also can be secured to the topsheet 306 with a bond 91 at a location 71 located inboard of the first location 70. The bond 91 and location 71 can be considered to be another primary bond and a first location. In the embodiment shown in FIG. 13, the outer cover 308 is further secured to the garment side surface of the fold 346 with a bond 351.

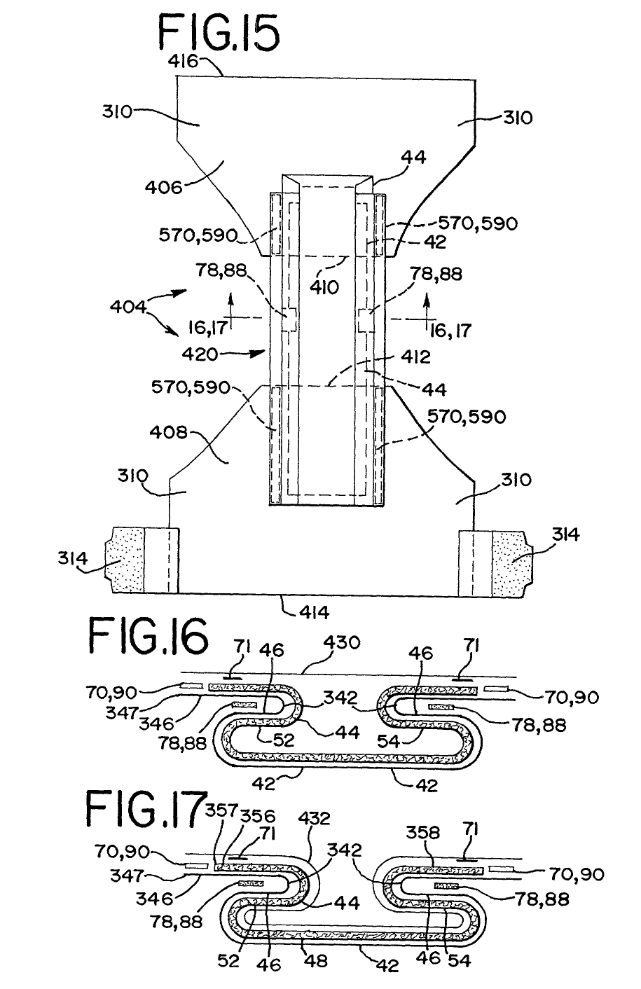

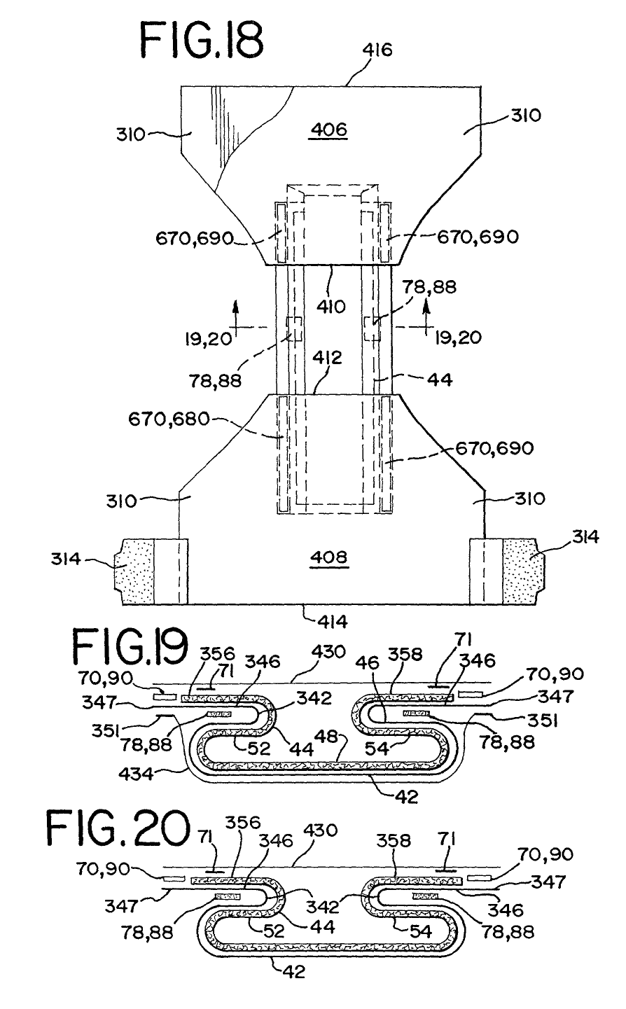

In the alternative embodiments shown in FIGS. 15-17, the absorbent element is secured to the inner, bodyside surface of the front and back panels 406, 408. In particular, the barrier layer 42 is secured to the body chassis with a primary bond 590 preferably at a location 570 along the garment side surface of the folds 346. As shown in FIG. 16, a top sheet 430, which in this embodiment is incorporated into the absorbent element, is substantially flat and is secured to the absorbent material as described above. Alternatively, as shown in FIG. 17, the top sheet 432 can be interfolded with the absorbent material. In the alternative embodiments shown in FIGS. 18-20, the absorbent element, and preferably the topsheet that is incorporated into the absorbent composite, is secured to the outer, garment side surface of the front and back panels 406, 408 with a primary bond 690 at a first location 670. Alternatively, the topsheet can be considered to be part of the body chassis, as it extends between the body panels, with the retention portion and barrier layer both secured to the topsheet with a primary bond at a first location as explained above. As shown in FIG. 19, the absorbent element can include an extensible outer cover 434, which is secured to the barrier layer as described above. Alternatively, as shown in FIG. 20, the outer cover is omitted. Alternatively, an extensible outer cover is preferably continuous and is disposed over the entire garment, wherein it is secured to the front and back panels and the absorbent element, and preferably the barrier layer thereof. It should be understood that in either of the embodiments of FIG. 15 or 18, the absorbent element can be fixedly detachably connected to the front and back body panels at the locations 670, such that the absorbent element can be removed and replaced with another element after each use.

The secondary bond material can comprise low tack adhesives and/or minimally thermal or ultrasonic bonding. Preferably, the secondary bond material comprises of a low tack adhesive that can be applied in specific locations on the folds of the absorbent element. One type of acceptable secondary bond material is a hot melt adhesive such as National Startch 34-5610 applied at very low basis weights to tack down the absorbent element and thereby keep it flat until expansion begins. Low add-on hot melt adhesives having low Tg values that soften when exposed to body temperatures will weaken and thereby give way as the absorbent material expands. Of course, it should be understood that other elements that detach when exposed to expansion forces also can be used. For example, hook and loop type fastening systems could be employed, preferably with an adhesion force less than the primary bonds, including any hook and loop type fastening systems used therefore.

As shown in FIGS. 2, 5 and 6, a liquid permeable liner 80, otherwise referred to as a topsheet, is disposed across the opening 16 formed in the chassis. The liner 80 can be attached to either of the chassis 4 or the absorbent element 40, and in particular the cover sheet 42. Alternatively, as shown in FIG. 3, the lateral edge portions 82 of the liner can simply be disposed between an inner surface 84 of the folds 46 of the cover sheet and a bodyside surface 86 of the absorbent material 44 so as to cover the absorbent material, and in particular, the bodyside surface 86 of the absorbent material exposed to the body of the user.

In operation, the absorbent material 44 initially has a relatively thin profile in a dry condition, prior to the introduction of liquid thereto, wherein the primary and second bonds 90, 88 connect the absorbent element to the chassis across substantially the entire width (W) of the absorbent element. During use, the user insults the absorbent garment with bodily exudates, including various bodily fluids and excrement, which passes through the opening 16 in the body chassis and through the liner 80, where it is introduced to the absorbent material 44. As the exudates are introduced to the absorbent material 44, the absorbent material 44 begins to swell or expand, as shown for example in FIG. 4. As the absorbent material 44 expands, it causes the secondary bonds 88 to break and the cover sheet 42 to detach from the chassis 4, or from itself, along the secondary bond regions 78. Preferably, the secondary bonds 88 are first broken at the outboard edge as the cover sheet 42 first begins to detach at the outboard side of the secondary bond region 78. The breaking of the secondary bonds 88 moves progressively inward as the absorbent material continues to expand.

In a fully expanded or swollen condition, the secondary bonds 88 are entirely broken between the cover sheet 42 and chassis 4, or between the cover sheet and itself. Of course, it should be understood that different levels of bond breaking and cover sheet detachment are achieved depending on the expanded condition of the absorbent material. The primary bonds 90 at the locations along the inboard edges of the cover sheet 42 maintain a firm attachment between the absorbent element 40 and the chassis 4, which in turn ensures that no liquid is allowed to escape. At the same time, the body chassis 4 is not pushed away from the body of the user as the absorbent material 44 expands, which again maintains a proper gasket between the chassis and the body of the user so as to prevent unwanted leakage while at the same time maintaining a snug fit between the chassis and the body of the user. In essence, the absorbent element works independently of the chassis, such that the user does not experience any discomfort or embarrassment.

Of course, it should be understood that the cover sheet of the absorbent element, with its folds, can be maintained in a relatively flat condition without the use of the secondary bonds. In this embodiment, the absorbent element unfolds, or expands, as the absorbent material expands away from the body of the user. The absorbent composite can include any of the various configurations of absorbent material.

The folds 52, 54 or layers 148, 150, 260, 252 of the preferred embodiments of the absorbent material increase the effective surface area available for the intake of exudates, and especially the fluid portions thereof, as explained above. As the fluid enters the folded or stacked absorbent material, it flows along the layers or folds of the material, such that it can be absorbed more quickly and such that the wicking of the fluid is improved. In addition, the folded or stacked absorbent material 44, 144, 244 allows for the use of relatively thin composite materials that can be unwound from rolls having greater relative lengths of absorbent material. In this way, the running times between roll changeovers can be extended.

In one preferred alternative embodiment, the cover sheet 42 and/or the outer cover 308 can be made of an extensible materials, such as necked, pleated (or micropleated) or creped nonwovens, including spunbond polypropylenes, bonded carded webs, or laminates of nonwovens and films that are necked, pleated or creped so as to allow the outer cover to extend with minimal force. For example, a suitable extensible material is a 60% necked, polypropylene spunbound having a basis weight of about 1.2 osy. The cover sheet 42 and outer cover 308 can also be made of nonwovens, films, or composites of films and nonwovens.

The cover sheet 42 and/or outer cover 308 also can be made of elastic materials such as Lycra® laminates, wherein the Lycra strands are laminated between two layers of nonwovens, stretch bonded laminates (SBL's), neck bonded laminates (NBL's) or elastomeric films or nonwovens. In this embodiment, the outer cover extends or stretches outwardly to accommodate the swelling absorbent material. In such an embodiment, the secondary bond regions may be optional, wherein the cover sheet maintains a thin profile when the absorbent material is not exposed to liquid, and wherein the cover sheet extends to accommodate the absorbent material when insulted with liquid.

The term “extensible” means capable of being extended, such that the cover sheet and/or outer cover provide a selected elongation when subjected to an applied tensile force. The cover sheet and/or the outer cover also are preferably capable of providing a selected, sustained deformation when subjected to an applied tensile force and then allowed to relax for a selected time period beginning immediately after removal of the tensile force. Preferably the sustained deformation is substantially permanent deformation. The selected elongation and sustained deformation preferably occur at least along the lateral cross-direction of the garment, although it should be understood that it also could occur along the longitudinal direction, or both. In one preferred embodiment, the cover sheet and/or outer cover is capable of providing an elongation of at least about 1 cm when subjected to a tensile force of 11.8 g/cm, and further provides a substantially permanent deformation of at least about 20% when subjected to a tensile force of 19.70 g/cm and is then allowed to relax under a zero applied stress for a period of 1 minute. Various extensible materials are further described in U.S. application Ser. No. 09/249,434 filed Feb. 12, 1999, entitled Expandable Cover Garment, the entire disclosure of which is hereby incorporated herein by reference.

The extensible cover sheet and/or outer cover can be substantially non-elastomeric, and does not have the stretch and retraction characteristics of natural rubber.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

What is claimed is:

1. An absorbent garment comprising:
a chassis; and
an absorbent element fixedly connected to said chassis at a first location and having a first portion detachably connected to at least one of a second portion of said absorbent element and said chassis at a second location, said absorbent element comprising an absorbent material expandable from at least a first condition to a second condition, wherein said first portion of said absorbent element detaches from said at least one of said second portion of said absorbent element and said chassis at at least a portion of said second location when said absorbent material expands to said second condition.

2. The invention of claim 1 wherein said absorbent element comprises a cover sheet fixedly connected to said chassis at said first location, wherein said cover sheet comprises said first and second portions of said absorbent element, wherein said first portion of said cover sheet is detachably connected to said at least one of said second portion of said cover sheet and said chassis at said second location, wherein said cover sheet supports said absorbent material.

3. The invention of claim 1 wherein said absorbent element is fixedly connected to said chassis at said first location with a primary bond, and wherein said first portion of said absorbent element is detachably connected to said at least one of said second portion of said absorbent element and said chassis at said second location with a secondary bond, wherein said secondary bond is weaker than said primary bond.

4. The invention of claim 1 wherein said absorbent material has a first and second side, and further comprising a topsheet disposed adjacent said first side of said absorbent material.

5. The invention of claim 1 wherein said second location is positioned laterally outboard from said first location.

6. The invention of claim 1 wherein said absorbent element has a longitudinal extent and wherein said first location extends longitudinally along at least a portion of said absorbent element.

7. The invention of claim 6 wherein said second location extends longitudinally along at least a portion of said absorbent element in a parallel relationship with said first location.

8. The invention of claim 1 wherein said absorbent element has a longitudinal extent, wherein said first location comprises a pair of laterally spaced, parallel and longitudinally extending primary locations, and wherein said second location comprises a pair of laterally spaced secondary locations.

9. The invention of claim 1 wherein said second location comprises a bonding region defined by a longitudinally extending length and a laterally extending width.

10. The invention of claim 6 wherein said absorbent element comprises opposite ends, wherein at least one of said ends is fixedly connected to said chassis.

11. The invention of claim 10 wherein said fixed connection between said at least one of said ends of said absorbent element and said chassis are spaced apart from said longitudinally extending first location.

12. The invention of claim 1 wherein said absorbent material comprises a first fold having opposite side edges and a second and third fold attached to said opposite side edges of said first fold respectively and extending inwardly in an overlying relationship with said first fold.

13. The invention of claim 1 wherein said absorbent material comprises a plurality of disconnected layers.

14. The invention of claim 2 wherein said absorbent material is not attached to said cover sheet.

15. The invention of claim 1 wherein said chassis comprises a top sheet and an extensible outer cover.

16. The invention of claim 1 wherein said chassis comprises a front and back panel, and wherein said absorbent element connects said front and back panels.

17. The invention of claim 16 wherein said absorbent element is connected to a bodyside surface of said front and back panels.

18. The invention of claim 4 wherein said topsheet is interfolded with said absorbent material.

19. The invention of claim 2 wherein said first portion of said cover sheet is detachably connected to said second portion of said cover sheet.

20. The invention of claim 19 wherein said first and second portions of said cover sheet comprise overlying folds of said cover sheet.

21. The invention of claim 2 wherein said first portion of said cover sheet is detachably connected to said chassis.

22. A method of absorbing exudates excreted from a user with an absorbent garment comprising:
providing a chassis and an absorbent element connected to said chassis at a first location, said absorbent element having a first portion detachably connected to at least one of a second portion of said absorbent element and said chassis at a second location, wherein said absorbent element comprises an absorbent material;
securing said chassis to a body of a user;
insulting said absorbent material with said exudates and thereby causing said absorbent material to expand; and
disconnecting said first portion of said absorbent element from said at least one of said second portion of said absorbent element and said chassis at at least a portion of said second location as said absorbent material expands while maintaining said connection between said absorbent element and said chassis at said first location.

23. The invention of claim 22 wherein said absorbent element comprises a cover sheet fixedly connected to said chassis at said first location and wherein said cover sheet comprises said first and second portions of said absorbent element, wherein said first portion of said cover sheet is detachably connected to at least one of said second portion of said cover sheet and said chassis at said second location, wherein said cover sheet supports said absorbent material.

24. The invention of claim 22 wherein said absorbent element is fixedly connected to said chassis at said first location with a primary bond, and wherein said first portion of said absorbent material is detachably connected to at least one of said second portion of said absorbent element and said chassis at said second location with a secondary bond, wherein said secondary bond is weaker than said primary bond.

25. The invention of claim 22 wherein said absorbent material has a first and second side, and further comprising a topsheet disposed adjacent said first side of said absorbent material.

26. The invention of claim 22 wherein said absorbent element has a longitudinal extent and wherein said second location is positioned laterally outboard from said first location.

27. The invention of claim 26 wherein said first location extends longitudinally along at least a portion of said absorbent element.

28. The invention of claim 27 wherein said second location extends longitudinally along at least a portion of said absorbent element in a parallel relationship with said first location.

29. The invention of claim 22 wherein said absorbent element has a longitudinal extent, wherein said first location comprises a pair of laterally spaced, parallel and longitudinally extending first locations, and wherein said second location comprises a pair of laterally spaced secondary locations.

30. The invention of claim 23 wherein said first portion of said cover sheet is detachably connected to said second portion of said cover sheet.

31. The invention of claim 23 wherein said first portion of said cover sheet is detachably connected to said chassis.

32. A method of assembling an absorbent garment comprising:
providing a chassis;
providing an absorbent element comprising an absorbent material;
bonding said absorbent element with a primary bond to said chassis at at least one primary bond region; and
bonding a first portion of said absorbent element to at least one of a second portion of said absorbent element and said chassis with a secondary bond at at least one secondary bond region, wherein said secondary bond is weaker than said primary bond.

33. The invention of claim 32 wherein said first and second bond regions are laterally spaced.

34. The invention of claim 32 wherein said primary bond region comprises a pair of laterally spaced, parallel and longitudinally extending primary bond regions, and wherein said secondary bond region comprises a pair of laterally spaced secondary bond regions.

35. The invention of claim 32 wherein said absorbent material comprises a first fold having opposite side edges and a second and third fold attached to said opposite side edges of said first fold respectively and extending inwardly in an overlying relationship with said first fold.

36. The invention of claim 32 farther comprising disposing a topsheet adjacent one side of said absorbent element.

37. The invention of claim 32 wherein said chassis comprises spaced apart front and back panels, and further comprising bonding said absorbent element to each of said front and back panels with said primary bond.

38. The invention of claim 32 wherein said absorbent element further comprises a cover sheet, wherein said cover sheet is bonded to said chassis with said primary bond at said primary bond region, wherein said cover sheet comprises said first and second portions of said absorbent element, wherein said first portion of is bonded to said second portion of said cover sheet with said secondary bond at said secondary bond region.

39. The invention of claim 32 wherein said absorbent element further comprises a cover sheet, wherein said cover sheet is bonded to said chassis with said primary bond at said primary bond region, and wherein said cover sheet comprises said first and second portions of said absorbent element, wherein said first portion of is bonded to said chassis with said secondary bond at said secondary bond region.

40. An absorbent garment comprising:
a chassis having an opening formed therein and comprising an outer surface; and
an absorbent element comprising an extensible cover sheet and an absorbent material supported by said cover sheet, wherein said extensible cover sheet is attached to said outer surface of said chassis on opposite sides of said opening such that said absorbent material covers said opening, wherein said cover sheet is extensible between at least a first and second position, and wherein said absorbent element comprises an absorbent material expandable from at least a first condition to a second condition, wherein said cover extends away from said chassis to said second position when said absorbent material expands to said second condition.

41. The invention of claim 40 wherein said cover sheet has a first portion detachably connected to at least one of a second portion of said cover sheet and said chassis.

42. The invention of claim 41 wherein said cover sheet is connected to said chassis with a primary bond and wherein said first portion of said cover sheet is detachably connected to at least one of said second portion of said cover sheet and said chassis with a secondary bond, wherein said primary bond is stronger than said secondary bond.

43. An absorbent garment comprising:
a chassis having an outer, garment side surface; and
an absorbent element having opposite side edges and a bodyside surface, wherein said bodyside surface of said absorbent element is connected to said outer surface of said chassis along a first location spaced laterally inward from said opposite side edges.

44. The invention of claim 43 wherein said first location comprises a pair of first locations spaced laterally inward from each of said opposite side edges respectively.

45. The invention of claim 43 wherein a first portion of said bodyside surface of said absorbent element is further connected to at least one of a second portion of said absorbent element and said outer surface of said chassis at a second location positioned between said first location and one of said opposite side edges of said absorbent element, wherein said absorbent element comprises an absorbent material expandable from at least a first condition to a second condition, and wherein said first portion of said absorbent element detaches from said at least one of said second portion and said chassis at at least a portion of said second location when said absorbent material expands to said second condition.

46. An absorbent garment comprising:
a reusable chassis having an outer surface; and
a disposable absorbent element detachably connected to said outer surface of said chassis.

47. An absorbent garment system comprising:
a reusable chassis having an outer surface; and
a plurality of disposable absorbent elements capable of being detachably connected to said outer surface of said chassis.

48. A method of providing absorbent protection to a user:
securing a reusable chassis to a body of the user, wherein said chassis has an outer surface, and wherein a disposable absorbent element is detachably connected to said outer surface of said chassis;
insulting said absorbent element with bodily exudates;
detaching said insulted disposable absorbent element from said outer surface of said chassis; and
attaching a second disposable absorbent element to said outer surface of said chassis.
